(19) United States
(12) Patent Application Publication
(10) Pub. No.: US 2025/0194930 A1
(43) Pub. Date: Jun. 19, 2025

(54) TERMINAL DEVICE AND MEASUREMENT METHOD

(71) Applicant: SHARP KABUSHIKI KAISHA, Sakai City, Osak (JP)

(72) Inventors: HIROYASU IWATSUKI, Sakai City, Osaka (JP); YOSHITOMO AKAGI, Sakai City, Osaka (JP); MASAHIKO NATSUME, Sakai City, Osaka (JP); KAZUTOSHI ISHIKAWA, Sakai City, Osaka (JP)

(21) Appl. No.: 18/849,783

(22) PCT Filed: Sep. 22, 2023
(86) PCT No.: PCT/JP2023/034416

§ 371 Date: Sep. 23, 2024

(30) Foreign Application Priority Data
Sep. 28, 2022 (JP) 2022-155298

(51) Int. Cl. A61B 5/00; G06T 7/00

(52) U.S. Cl. CPC A61B 5/0077; G06T 7/0012

(57) ABSTRACT

A terminal device that can appropriately execute non-contact vital sensing while reducing a burden on a measurer and a subject to be measured is provided. A terminal device includes a capturing device, an acquisition unit configured to acquire a captured image of a subject to be measured, and a measurement unit configured to measure biological information of the subject to be measured. The measurement unit performs processing of measuring the biological information based on a facial image of the subject to be measured and switches, if failing in measurement of the biological information based on a facial image, from the processing of measuring the biological information based on the facial image of the subject to be measured to processing of measuring the biological information based on a hand image or a foot image of the subject to be measured.

### TECHNICAL FIELD

The present invention relates to a terminal device and a measurement method by the terminal device.

### BACKGROUND ART

A known technique (non-contact vital sensing) detects biological information of a subject to be measured based on a captured image in which the subject to be measured is captured. For example, PTL 1 discloses a measurement method of a pulse wave velocity of a human body. The measurement method includes simultaneously imaging two sites different from each other from among a plurality of sites of the human body in a non-contact state and calculating the pulse wave velocity of the human body based on a time difference between pulse waves at the two sites. PTL 2 discloses a biological information analysis system that outputs biological information while suppressing external noise by analyzing a difference signal of a fundamental wave indicating a temporal change of a representative color between adjacent regions.

### CITATION LIST

### Patent Literature

PTL 1: JP 6072893 B
PTL 2: JP 6329696 B

### SUMMARY OF INVENTION

### Technical Problem

However, in the above-described techniques, in order to simultaneously capture different sites of the subject to be measured, the different sites need to be simultaneously captured in a captured image. Therefore, it can be a burden on the subject to be measured.

In a method of measuring biological information of a subject to be measured based on a facial image of the subject to be measured captured by a terminal device, if the face of the subject to be measured is suntanned or made-up, measurement of the biological information may fail. In such a case, the measurer measures the biological information using a device (such as a sphygmomanometer) in contact with the subject to be measured in place of a terminal device. Therefore, there is a problem that non-contact vital sensing cannot be achieved. Switching a measuring instrument to another measuring instrument can also be a burden on the measurer.

One aspect of the present invention is to provide a terminal device that can appropriately execute non-contact vital sensing while reducing a burden on a measurer and a subject to be measured.

### Solution to Problem

To solve the above problem, a terminal device according to one aspect of the present invention includes: a capturing device; an acquisition unit configured to acquire a captured image of a subject to be measured captured by the capturing device; and a measurement unit configured to measure biological information of the subject to be measured based on an image of a predetermined site of the subject to be measured in the captured image. The measurement unit performs processing of measuring the biological information based on a facial image of the subject to be measured as an image of the predetermined site and switches, if failing in measurement of the biological information based on the facial image, from the processing of measuring the biological information based on the facial image of the subject to be measured to processing of measuring the biological information based on a hand image or a foot image of the subject to be measured.

To solve the above problem, a measurement method according to one aspect of the present invention is a measurement method by a terminal device including a capturing device. The measurement method includes: measuring, by the terminal device, biological information of the subject to be measured based on a facial image of the subject to be measured; determining, by the terminal device, whether measurement of the biological information based on the facial image in the measuring has failed; and measuring, by the terminal device, the biological information based on a hand image or a foot image of the subject to be measured if the measurement of the biological information based on the facial image has failed.

### Advantageous Effects of Invention

One aspect of the present invention can appropriately execute non-contact vital sensing while reducing a burden on a measurer and a subject to be measured.

### BRIEF DESCRIPTION OF DRAWINGS

FIG. 1 is a block diagram illustrating a schematic configuration of a terminal device according to the present embodiment.

FIG. 2 is a view illustrating an example of an image displayed by a display device.

FIG. 3 is a view illustrating another example of an image displayed by the display device.

FIG. 4 is a flowchart showing an example of an operation example of the terminal device.

### DESCRIPTION OF EMBODIMENTS

### First Embodiment

### 1. Schematic Configuration of Terminal Device 100

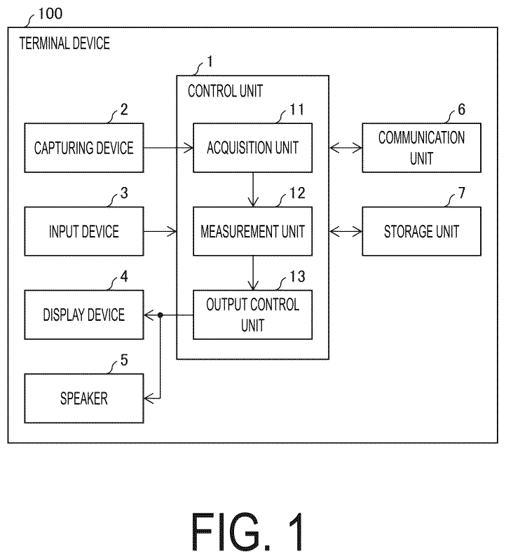

FIG. 1 is a block diagram illustrating a schematic configuration of a terminal device 100 according to the present embodiment. As illustrated in FIG. 1, the terminal device 100 includes a control unit 1, a capturing device 2, an input device 3, a display device 4, a speaker 5 (voice output device), a communication unit 6, and a storage unit 7. The terminal device 100 is a device used to measure biological information of a subject to be measured based on a captured image of the subject to be measured (i.e., in a non-contact manner). Examples of the item of biological information include a blood pressure and a pulse of the subject to be measured but are not limited thereto. The terminal device 100 has an image acquisition function and an image display function and may be, for example, a smartphone or a tablet terminal.

A measurer measures biological information of the subject to be measured by using the terminal device 100 and continuing capturing the subject to be measured while confirming an image including the subject to be measured displayed on a screen of the display device 4. The terminal device 100 is used when a caregiver (measurer) measures biological information of a care receiver (subject to be measured) at a care site, for example.

Each function of the terminal device 100 described below can also be implemented by installing a program into a general-purpose computer. The program can also be called application software. Hereinafter, an example in which the terminal device 100 is a smartphone in which the program is installed will be described.

The control unit 1 integrally controls each unit of the terminal device 100. The capturing device 2 captures an image. The input device 3 receives an input operation to the terminal device 100. The display device 4 has the screen, and displays, on the screen, an image captured by the capturing device 2. The speaker 5 outputs a predetermined voice based on an instruction by the control unit 1. The communication unit 6 transmits and receives various data between an external communication device and the control unit 1. The storage unit 7 stores various data handled by the terminal device 100. The input device 3 may be a touchscreen, and in this case, the input device 3 is configured integrally with the display device 4.

The control unit 1 includes an acquisition unit 11, a measurement unit 12, and an output control unit 13.

The acquisition unit 11 acquires a captured image of a subject to be measured captured by the capturing device 2, as information for measuring biological information of the subject to be measured. The captured image may be a moving image of the subject to be measured or a still image of the subject to be measured. The acquisition unit 11 may acquire a moving image as a captured image when performing measurement based on a change in a capturing target and may acquire a still image as a captured image when performing measurement not based on a change in the capturing target.

The measurement unit 12 measures biological information of the subject to be measured based on an image (a facial image, a hand image, a foot image, or the like) of a predetermined site of the subject to be measured in a captured image. As a measurement method of biological information based on an image of a predetermined site, a known technique can be used. For example, the measurement unit 12 calculates biological information using a determination table corresponding to a predetermined site based on image data (information of color tone or the like) of the predetermined site. The determination table includes parameters used in a determination expression for calculating biological information from image data of a predetermined site. An external server in place of the measurement unit 12 may measure biological information of the subject to be measured. In this case, the captured image acquired by the acquisition unit 11 is transmitted to the external server via the communication unit 6.

The measurement unit 12 sets a predetermined region in a captured image as a measurement region for measuring biological information. Here, the measurement unit 12 may recognize, using a known technique, the predetermined site (face, hand, foot, or the like) included in the captured image. For example, the acquisition unit 11 specifies a position, an orientation, and the like of the predetermined site included in the captured image. If the position of the predetermined site specified by the acquisition unit 11 is out of the measurement region, the measurement unit 12 does not need to measure the biological information of the subject to be measured. If the position of the predetermined site is out of the measurement region, the predetermined site of the subject to be measured is guided to a measurement region (predetermined position) by a guide function of the terminal device 100 described later, and the predetermined site of the subject to be measured is fixed in the measurement region. This enables the measurement unit 12 to measure the biological information of the subject to be measured based on an image of the predetermined site.

The measurement unit 12 determines whether measurement of biological information based on an image of a predetermined site has failed. Specifically, if the measurement result of the biological information is an abnormal value (not within the normal range), the measurement unit 12 determines that the measurement of the biological information has failed.

In the present embodiment, the measurement unit 12 first performs processing of measuring biological information of the subject to be measured based on a facial image of the subject to be measured (hereinafter, called first measurement processing). Then, in a case of having failed in measurement of the biological information based on the facial image, the measurement unit 12 switches from the first measurement processing to processing of measuring the biological information based on a hand image or a foot image of the subject to be measured (hereinafter, called second measurement processing). Specifically, the measurement unit 12 switches the determination table used for measurement of the biological information from a determination table corresponding to the face to a determination table corresponding to the hand or foot. Such switching from the first measurement processing to the second measurement processing does not require a user's instruction (input operation to the input device 3 or the like), and is automatically executed with a failure in measurement of the biological information based on the facial image as a trigger. Examples of the cause of failure in measurement of the biological information based on the facial image include suntan or makeup of the face of the subject to be measured.

The output control unit 13 displays a captured image onto the screen of the display device 4. The output control unit 13 causes an index image that guides the position of a predetermined site of the subject to be measured to a predetermined position to be superimposed and displayed on the captured image (image including the subject to be measured).

In the present embodiment, when the measurement unit 12 performs the first measurement processing, the output control unit 13 causes a face index image that guides the position of the face of the subject to be measured to a predetermined position to be superimposed and displayed on the captured image. The face index image may be a contour guide having a human shape including a face portion and a shoulder portion. Then, if the measurement unit 12 fails in measurement of the biological information based on the facial image (i.e., when the measurement unit 12 switches from the first measurement processing to the second measurement processing), the output control unit 13 outputs information for guiding the measurer or the subject to be measured and thus causing the measurer to capture a hand image or a foot image of the subject to be measured.

For example, if the measurement unit 12 fails in measurement of the biological information based on the facial image, the output control unit 13 may cause a hand index image or a foot index image (index image) that guides the position of the hand or the foot of the subject to be measured to a predetermined position to be superimposed and displayed on the captured image. Due to this, the measurer is guided to align the position of the hand or the foot with the hand index image or the foot index image, thus allowing for continuing capturing with the hand or the foot not misaligning from the predetermined position. The hand index image or the foot index image may be a contour guide of the hand or the foot, respectively. Use of the hand index image or the foot index image as a contour guide of the hand or the foot, respectively, can make the measurer aware that the terminal device 100 should not be moved for capturing the hand image or the foot image of the subject to be measured.

The index image may be a figure (e.g., a cross mark) indicating a position to be the center of a predetermined site of the subject to be measured when the predetermined site of the subject to be measured is aligned with a predetermined position on the screen of the display device 4. The index image may be a figure indicating a position of a part (e.g., a nose, a mouth, an ear, and the like when the predetermined site is a face) constituting a predetermined site of the subject to be measured.

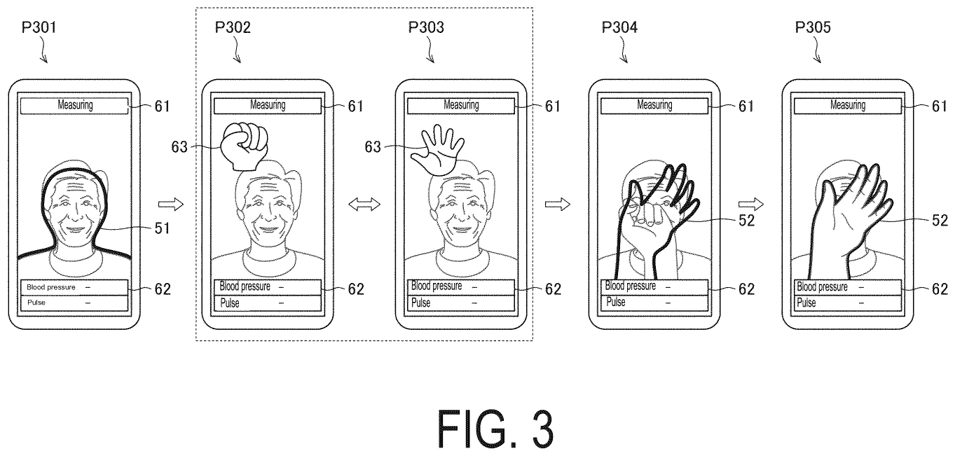

If the measurement unit 12 fails in measurement of the biological information based on the facial image, the output control unit 13 may cause a notification image for notifying that the measurement unit 12 has switched from the first measurement processing to the second measurement processing to be superimposed and displayed on the captured image. This can notify the measurer that the measurement based on the facial image has failed and the first measurement processing is switched to the second measurement processing. For example, the output control unit 13 may cause an image of a hand or a foot as the notification image to be displayed in order to notify the measurer of start of the second measurement processing (see FIG. 3).

If the measurement unit 12 fails in measurement of the biological information based on the facial image, the output control unit 13 may cause the speaker 5 to output a voice that guides the subject to be measured and thus causes the subject to be measured to turn the palm in the same orientation as a face orientation. This enables the subject to be measured to take a naturally measurable posture (turn the palm to the capturing device 2 with the palm falling within the capturing range of the capturing device 2) in accordance with the voice. The voice may guide the subject to be measured and thus cause the subject to be measured to fix the hand with respect to the face. This can guide the subject to be measured to fix the palm at a predetermined position. In this manner, the terminal device 100 guides the subject to be measured to a state where the palm is measurable with reference to the orientation or the position of the face in the first measurement processing. Therefore, the voice that guides can cause the palm, which is the next measurement target, to be brought to the face or nearby, which is the previous measurement target. Therefore, the terminal device 100 can easily fit the palm, which is the next measurement target, in the capturing range of the capturing device 2. The subject to be measured can easily take a measurable posture.

For example, the voice may guide the subject to be measured and thus cause the subject to be measured to put a thumb of a right hand or a left hand on a chin and put an index finger of a right hand or a left hand on a right ear or a left ear, respectively. Alternatively, the voice may guide the subject to be measured and thus cause the subject to be measured to spread fingers while placing a back of a hand on a nose.

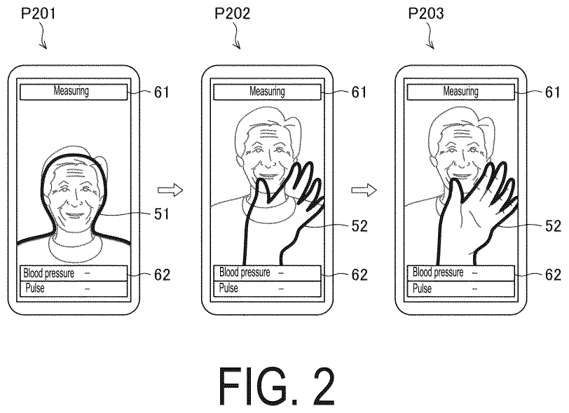

The output control unit 13 may superimpose, on the captured image, and display a character image 61 including character information indicating a situation related to measurement (see FIGS. 2 and 3). The output control unit 13 may further superimpose, on the captured image, and display a biological information item that is a measurement target of the measurement unit 12 and a measurement result image 62 indicating the measurement result (see FIGS. 2 and 3).

### 2. Guide Function of Terminal Device

Next, a guide function of the terminal device 100 that guides the measurer or the subject to be measured and thus causes the measurer to capture a hand image of the subject to be measured when failing in measurement of the biological information based on the facial image will be described with reference to FIGS. 2 and 3.

FIG. 2 is a view illustrating an example of an image displayed by the display device 4 (when the measurement unit 12 fails in measurement of biological information based on a facial image and measures biological information based on a hand image in place of the facial image). A display image P201 in FIG. 2 indicates an image displayed by the display device 4 during execution of the first measurement processing. A display image P202 in FIG. 2 indicates an image displayed by the display device 4 after end of the first measurement processing and before start of the second measurement processing. A display image P203 in FIG. 2 indicates an image displayed by the display device 4 during execution of the second measurement processing.

As illustrated in the display image P201, during the execution of the first measurement processing, the output control unit 13 causes a face index image 51 (contour guide having a human shape including the face portion and the shoulder portion) to be displayed at a predetermined position on the screen of the display device 4. The measurer continues capturing the subject to be measured and thus causes the face portion and the shoulder portion of the subject to be measured to match the face index image 51.

As illustrated in the display image P202, after the end of the first measurement processing and before the start of the second measurement processing, the output control unit 13 causes a hand index image 52 (hand contour guide) to be displayed at a predetermined position on the screen of the display device 4. The measurer confirms that the hand index image 52 is displayed, and recognizes that the measurement unit 12 fails in measurement of the biological information based on the facial image and measures the biological information based on the hand image in place of the facial image.

After the end of the first measurement processing and before the start of the second measurement processing, the output control unit 13 causes the speaker 5 to output a voice (voice “Put the thumb of your left hand on your chin and the index finger of your left hand on your left ear”) that guides the subject to be measured and thus causes the subject to be measured to put the thumb of the left hand on the chin and the index finger of the left hand on the left ear. The subject to be measured puts the thumb of the left hand on the chin and the index finger of the left hand on the left ear in accordance with the voice. This enables the subject to be measured to naturally turn the palm to the capturing device 2 and fix the palm at a predetermined position. The measurer adjusts the capturing region by moving the terminal device 100 and thus causes the palm of the subject to be measured to match the hand index image 52. Due to this, since the palm of the subject to be measured is fixed in the measurement region, the measurement unit 12 can execute the second measurement processing.

As illustrated in the display image P203, even during the execution of the second measurement processing, the output control unit 13 causes the hand index image 52 to be displayed at a predetermined position on the screen of the display device 4. The measurer continues capturing the subject to be measured and thus causes the palm of the subject to be measured to match the hand index image 52.

As described above, by displaying the hand index image 52, the terminal device 100 can guide the measurer and thus causes the palm of the subject to be measured to match the hand index image 52. Therefore, it is possible to cause the measurer to continue capturing with the palm not deviating from the predetermined position. By outputting the above-described voice, the terminal device 100 can cause the subject to be measured to naturally turn the palm to the capturing device 2 and fix the palm at a predetermined position.

FIG. 3 is a view illustrating another example of an image displayed by the display device 4 (when the measurement unit 12 fails in measurement of the biological information based on a facial image and measures the biological information based on a hand image in place of the facial image). A display image P301 in FIG. 3 illustrates an image similar to the display image P201 of FIG. 2, which is displayed by the display device 4 during the execution of the first measurement processing. Display images P302 to P304 in FIG. 3 illustrate images displayed by the display device 4 after the end of the first measurement processing and before the start of the second measurement processing. A display image P305 in FIG. 3 illustrates an image similar to the display image P203 of FIG. 2, which is displayed by the display device 4 during the execution of the second measurement processing.

As illustrated in the display images P302 and P303, after the end of the first measurement processing and before the start of the second measurement processing, the output control unit 13 may cause a notification image 63 for notifying that the measurement unit 12 has switched from the first measurement processing to the second measurement processing to be displayed at a predetermined position on the screen of the display device 4. In the example illustrated in FIG. 3, the notification image 63 is an animation of repeating a gesture of clenching the first and a gesture of unclenching the first. The measurer confirms the notification image 63, and recognizes that the measurement unit 12 fails in measurement of the biological information based on the facial image and measures the biological information based on the hand image in place of the facial image.

After displaying the notification image 63 for a predetermined period of time, the output control unit 13 causes the hand index image 52 to be displayed at a predetermined position on the screen of the display device 4 as illustrated in the display image P304. The output control unit 13 causes the speaker 5 to output a voice (voice “Place the back of your hand on your nose and open your hand”) that guides the subject to be measured and thus causes the subject to be measured to spread fingers while placing the back of the hand on the nose. The subject to be measured first puts the back of the hand on the nose in a state where the hand is closed as illustrated in the display image P304 in accordance with the voice. Next, the subject to be measured opens the hand as illustrated in the display image P305. This enables the subject to be measured to naturally turn the palm to the capturing device 2 and fix the palm at the same position as the face position. The measurer adjusts the capturing region by moving the terminal device 100 and thus causes the palm of the subject to be measured to match the hand index image 52. Due to this, since the palm of the subject to be measured is fixed in the measurement region, the measurement unit 12 can execute the second measurement processing.

As described above, the terminal device 100 displays the hand index image 52 and thus can guide the measurer to cause the palm of the subject to be measured to match the hand index image 52. Therefore, it is possible to cause the measurer to continue capturing with the palm not deviating from the predetermined position. By displaying the notification image 63, the terminal device 100 can notify the measurer that the measurement unit 12 fails in measurement of the biological information based on the facial image and measures the biological information based on the hand image in place of the facial image. By outputting the above-described voice, the terminal device 100 can cause the subject to be measured to naturally turn the palm to the capturing device 2 and fix the palm at the same position as the face position. Since the palm is fixed at the same position as the face position, the measurer can capture the palm of the subject to be measured with little movement of the terminal device 100 when switching from the first measurement processing to the second measurement processing.

As illustrated in FIGS. 2 and 3, the output control unit 13 may display the character image 61 at an upper part of the screen of the display device 4 and the measurement result image 62 at a lower part of the screen of the display device 4 during display of the display image. Specifically, the output control unit 13 may cause character information indicating that the measurement of the biological information has not been completed yet (text of “Measuring”) to be displayed on the character image 61 until the measurement by the measurement unit 12 is completed. The output control unit 13 may indicate, on the measurement result image 62, information indicating that any measurement result of biological information has not yet been obtained until the measurement by the measurement unit 12 is completed. The output control unit 13 may display the measurement result on the measurement result image 62 sequentially from the item of the biological information whose measurement has been completed.

### 3. Operation Example of Terminal Device 100

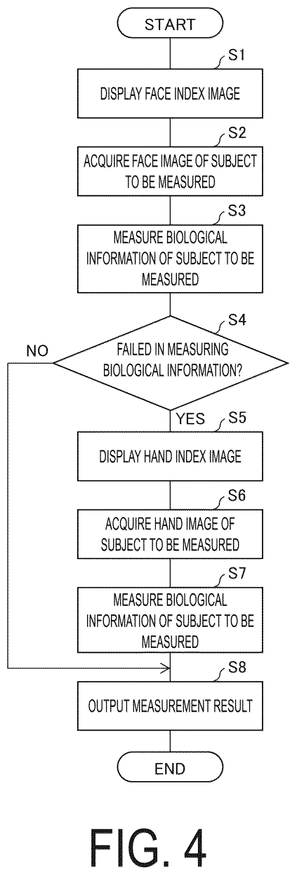

FIG. 4 is a flowchart showing an example of the operation of the terminal device 100. An operation example for the terminal device 100 to switch from the first measurement processing to the second measurement processing will be described below with reference to FIG. 4.

First, the output control unit 13 causes the face index image 51 to be superimposed and displayed on the captured image (S1). The measurer adjusts the capturing region by moving the terminal device 100 and thus causing the face portion and the shoulder portion of the subject to be measured to match the face index image 51. This fixes the face of the subject to be measured in the measurement region.

Next, the acquisition unit 11 acquires a facial image of the subject to be measured in the measurement region in the captured image captured by the capturing device 2, as information for measuring biological information of the subject to be measured (S2). Next, the measurement unit 12 measures the biological information of the subject to be measured based on the facial image (first measurement step S3). That is, the measurement unit 12 executes the first measurement processing.

Next, the measurement unit 12 determines whether measurement of biological information based on the facial image has failed (determination step S4). Specifically, the measurement unit 12 determines whether or not the measurement result obtained by the first measurement processing is an abnormal value. If the measurement unit 12 has succeeded in the measurement of the biological information based on the facial image (No in S4), the output control unit 13 outputs a measurement result of the biological information measured based on the facial image (S8). If the measurement unit 12 fails in the measurement of the biological information based on the facial image (Yes in S4), the output control unit 13 causes the hand index image 52 to be superimposed and displayed on the captured image (S5). The measurer confirms that the hand index image 52 is displayed, and recognizes that the measurement unit 12 fails in measurement of the biological information based on the facial image and measures the biological information based on the hand image in place of the facial image. The measurer then adjusts the capturing region by moving the terminal device 100 and thus causing the palm of the subject to be measured to match the hand index image 52. This fixes the palm of the subject to be measured in the measurement region.

In S5, the output control unit 13 may cause the speaker 5 to output a voice that guides the subject to be measured and thus causes the subject to be measured to turn the palm in the same orientation as the face orientation. The subject to be measured turns the palm in the same orientation as the face orientation in accordance with the voice. The measurer may adjust the capturing region and thus causes the palm of the subject to be measured facing the capturing device 2 to match the hand index image 52 in this manner.

Next, the acquisition unit 11 acquires a hand image of the subject to be measured in the measurement region in the captured image captured by the capturing device 2, as information for measuring biological information of the subject to be measured (S6). Next, the measurement unit 12 measures the biological information of the subject to be measured based on the hand image (second measurement step S7). That is, the measurement unit 12 executes the second measurement processing. Next, the output control unit 13 outputs a measurement result of the biological information measured based on the hand image (S8).

According to the above configuration, if the measurement of the biological information based on the facial image of the subject to be measured has failed, the terminal device 100 switches from the first measurement processing to the second measurement processing. Therefore, even if the measurement of the biological information has failed, the biological information of the subject to be measured can be measured based on the image of the hand or foot of the subject to be measured (having low possibility of failure due to suntan, makeup, or the like). Therefore, the terminal device 100 can more reliably achieve non-contact vital sensing. The burden on the measurer can be reduced. The terminal device 100 first attempts measurement of the biological information based on the facial image without causing the subject to be measured to take a specific posture such as raising a hand and turning the hand to the camera. Therefore, an unnecessary increase in measurement time can be prevented.

If failing in the measurement based on the facial image, the terminal device 100 performs the measurement based on the hand image or the foot image. Therefore, it is not necessary to simultaneously capture different sites in the captured image, and the burden on the subject to be measured can be reduced.

The above configuration can appropriately execute non-contact vital sensing while reducing a burden on a measurer and a subject to be measured. Such an effect also contributes to achievement of, for example, “Good Health and Well-Being”, Goal 3 of Sustainable Development Goals (SDGs) proposed by the United Nations.

### Implementation Example by Software

The function of the terminal device 100 (hereinafter, called “device”) is a program configured to cause a computer to function as the device, and can be implemented by a program configured to cause a computer to function as each control block of the device (particularly, each unit included in the control unit 1).

In this case, the device includes a computer having at least one control device (e.g., a processor) and at least one storage device (e.g., a memory) as hardware for executing the program. By executing the program by the control device and the storage device, the functions described in the above embodiment are implemented.

The program may be recorded in one or a plurality of non-transitory computer-readable recording media. This recording medium may be or may not be included in the device. In the latter case, the program may be supplied to the device via any wired or wireless transmission medium.

Some or all of the functions of the control blocks can also be implemented by a logic circuit. For example, an integrated circuit in which a logic circuit functioning as the control blocks is formed is also included in the scope of the present invention. In addition to this, for example, the functions of the control blocks can be implemented by a quantum computer.

Each processing described in the above embodiment may be executed by artificial intelligence (AI). In this case, the AI may operate on the control device, or may operate on another device (e.g., an edge computer, a cloud server, or the like).

### Supplement

A terminal device according to a first aspect of the present invention includes: a capturing device; an acquisition unit configured to acquire a captured image of a subject to be measured captured by the capturing device; and a measurement unit configured to measure biological information of the subject to be measured based on an image of a predetermined site of the subject to be measured in the captured image. The measurement unit performs processing of measuring the biological information based on a facial image of the subject to be measured as an image of the predetermined site and switches, if failing in measurement of the biological information based on the facial image, from the processing of measuring the biological information based on the facial image of the subject to be measured to processing of measuring the biological information based on a hand image or a foot image of the subject to be measured.

In a known method of measuring biological information of a subject to be measured based on a facial image of the subject to be measured captured by a terminal device (non-contact vital sensing), if the face of the subject to be measured is suntanned or made-up, measurement of the biological information may fail. In such a case, the measurer measures the biological information using a device (such as a sphygmomanometer) in contact with the subject to be measured in place of a terminal device. Therefore, there is a problem that non-contact vital sensing cannot be achieved. Switching a measuring instrument to another measuring instrument can also be a burden on the measurer.

According to the above configuration, in a case of having failed in measurement of the biological information based on the facial image of the subject to be measured, the terminal device switches from the processing of measuring the biological information based on the facial image of the subject to be measured to the processing of measuring the biological information based on the hand image or the foot image of the subject to be measured. Therefore, even if the measurement of the biological information has failed, the biological information of the subject to be measured can be measured based on the image of the hand or foot of the subject to be measured (having low possibility of failure due to suntan, makeup, or the like). Therefore, the terminal device can more reliably achieve non-contact vital sensing. The burden on the measurer can be reduced. The terminal device first attempts measurement of the biological information based on the facial image without causing the subject to be measured to take a specific posture such as raising the palm and turning the hand to the capturing device. Therefore, an unnecessary increase in measurement time can be prevented.

In the known method of simultaneously capturing different sites of the subject to be measured and measuring biological information of the subject to be measured, in order to simultaneously capture different sites, the different sites need to be simultaneously captured in a captured image. Therefore, it can be a burden on the subject to be measured.

According to the above configuration, if the measurement based on the facial image has failed, the terminal device performs the measurement based on the hand image or the foot image. Therefore, it is not necessary to simultaneously capture different sites in the captured image, and the burden on the subject to be measured can be reduced.

A terminal device according to a second aspect of the present invention may further include, in addition to the first aspect described above, an output control unit configured to output information for guiding a measurer or the subject to be measured and thus causing the measurer to capture a hand image or a foot image of the subject to be measured if the measurement unit fails in measurement of the biological information based on the facial image.

When the measurement based on the facial image is switched to the measurement based on the hand image or the foot image, the subject to be measured needs to change the posture in order to fit the palm or the sole in the captured image.

According to the above configuration, the terminal device outputs information for guiding the measurer or the subject to be measured and thus causing the measurer to capture a hand image or a foot image of the subject to be measured. This enables the measurer to recognize that the measurement based on the facial image has failed and that the measurement processing based on the facial image is switched to the measurement processing based on the hand image or the foot image, or the subject to be measured can take a measurable posture by himself/herself.

A terminal device according to a third aspect of the present invention may further include, in addition to the second aspect described above, a display device configured to display an image including the subject to be measured. If the measurement unit fails in measurement of the biological information based on the facial image, the output control unit may cause an index image that guides a position of a hand or a foot of the subject to be measured to a predetermined position to be superimposed and displayed on an image including the subject to be measured.

According to the above configuration, the terminal device displays an index image that guides the position of the hand or foot of the subject to be measured to a predetermined position. This causes the measurer to be guided to match the position of the hand or the foot with the index image, and thus the measurer can continue capturing with the hand or the foot not deviating from the predetermined position.

In a terminal device according to a fourth aspect of the present invention in addition to the third aspect described above, the index image may be a contour guide of a hand or a foot.

According to the above configuration, the terminal device can make the measurer aware that the terminal device should not be moved for capturing the hand image or the foot image of the subject to be measured.

In the terminal device according to a fifth aspect of the present invention in addition to the fourth aspect described above, if the measurement unit fails in measurement of the biological information based on the facial image, the output control unit may cause a notification image for notifying that the processing of measuring the biological information based on a facial image of the subject to be measured is switched to the processing of measuring the biological information based on a hand image or a foot image of the subject to be measured to be superimposed and displayed on an image including the subject to be measured.

According to the above configuration, the terminal device can notify the measurer that the measurement based on the facial image has failed and the measurement processing based on the facial image is switched to the measurement processing based on the hand image or the foot image.

A terminal device according to a sixth aspect of the present invention may further include, in addition to the second aspect described above, a voice output device. If the measurement unit fails in measurement of the biological information based on the facial image, the output control unit may cause the voice output device to output a voice that guides the subject to be measured and thus causes the subject to be measured to turn a palm in a same orientation as a face orientation.

According to the above configuration, the terminal device can guide the subject to be measured to turn the palm to the capturing device.

In a terminal device according to a seventh aspect of the present invention in addition to the sixth aspect described above, the voice may guide the subject to be measured and thus cause the subject to be measured to fix a hand with respect to a face.

According to the above configuration, the terminal device can guide the subject to be measured to fix the palm to a predetermined position.

In a terminal device according to an eighth aspect of the present invention in addition to the seventh aspect described above, the voice may guide the subject to be measured and thus cause the subject to be measured to put a thumb of a right hand or a left hand on a chin and put an index finger of a right hand or a left hand on a right ear or a left ear, respectively.

According to the above configuration, the terminal device can cause the subject to be measured to naturally turn the palm to the capturing device and fix the palm at a predetermined position.

In a terminal device according to a ninth aspect of the present invention in addition to the seventh aspect described above, the voice may guide the subject to be measured and thus cause the subject to be measured to spread fingers while placing a back of a hand on a nose.

According to the above configuration, the terminal device can cause the subject to be measured to naturally turn the palm to the capturing device and fix the palm at the same position as the face position. Since the palm is fixed at the same position as the face position, the measurer can capture the palm of the subject to be measured with little movement of the terminal device when switching from the measurement processing based on the facial image to the measurement processing based on the hand image or the foot image.

A measurement method according to a tenth aspect of the present invention is a measurement method by a terminal device including a capturing device. The measurement method includes: measuring, by the terminal device, biological information of the subject to be measured based on a facial image of the subject to be measured; determining, by the terminal device, whether measurement of the biological information based on the facial image in the measuring has failed; and measuring, by the terminal device, the biological information based on a hand image or a foot image of the subject to be measured if the measurement of the biological information based on the facial image has failed.

The terminal device according to each aspect of the present invention may be implemented by a computer. In this case, a control program of a terminal device that causes a computer to implement the terminal device by causing the computer to operate as each unit (software element) included in the terminal device and a computer-readable recording medium recording the control program are included in the scope of the present invention.

The present invention is not limited to each of the above-described embodiments. It is possible to make various modifications within the scope of the claims. An embodiment obtained by appropriately combining technical elements disclosed in different embodiments also falls within the technical scope of the disclosure. Furthermore, novel technical features can be formed by combining the technical approaches disclosed in each of the embodiments.

### REFERENCE SIGNS LIST

1 Control unit
2 Capturing device
4 Display device
5 Speaker (voice output unit)
11 Acquisition unit
12 Measurement unit
13 Output control unit
51 Face index image
52 Hand index image
63 Notification image
100 Terminal device

1. A terminal device, comprising:
a capturing device;
an acquisition unit configured to acquire a captured image of a subject to be measured captured by the capturing device; and
a measurement unit configured to measure biological information of the subject to be measured based on an image of a predetermined site of the subject to be measured in the captured image,
wherein the measurement unit
performs processing of measuring the biological information based on a facial image of the subject to be measured as an image of the predetermined site and
switches, if failing in measurement of the biological information based on the facial image, from the processing of measuring the biological information based on the facial image of the subject to be measured to processing of measuring the biological information based on a hand image or a foot image of the subject to be measured.

2. The terminal device according to claim 1 further comprising an output control unit configured to output information for guiding a measurer or the subject to be measured and thus causing the measurer to capture a hand image or a foot image of the subject to be measured if the measurement unit fails in measurement of the biological information based on the facial image.

3. The terminal device according to claim 2 further comprising
a display device configured to display an image including the subject to be measured,
wherein if the measurement unit fails in measurement of the biological information based on the facial image, the output control unit causes an index image that guides a position of a hand or a foot of the subject to be measured to a predetermined position to be superimposed and displayed on an image including the subject to be measured.

4. The terminal device according to claim 3, wherein the index image is a contour guide of a hand or a foot.

5. The terminal device according to claim 3, wherein if the measurement unit fails in measurement of the biological information based on the facial image, the output control unit causes a notification image for notifying that the processing of measuring the biological information based on a facial image of the subject to be measured is switched to the processing of measuring the biological information based on a hand image or a foot image of the subject to be measured to be superimposed and displayed on an image including the subject to be measured.

6. The terminal device according to claim 2 further comprising
a voice output device,
wherein if the measurement unit fails in measurement of the biological information based on the facial image, the output control unit causes the voice output device to output a voice that guides the subject to be measured and thus causes the subject to be measured to turn a palm in the same orientation as a face orientation.

7. The terminal device according to claim 6, wherein the voice guides the subject to be measured and thus causes the subject to be measured to fix a hand with respect to a face.

8. The terminal device according to claim 7, wherein the voice guides the subject to be measured and thus causes the subject to be measured to put a thumb of a right hand or a left hand on a chin and put an index finger of the right hand or the left hand on a right ear or a left ear, respectively.

9. The terminal device according to claim 7, wherein the voice guides the subject to be measured and thus causes the subject to be measured to spread fingers while placing a back of the hand on a nose.

10A measurement method by a terminal device including a capturing device, the measurement method comprising:
measuring, by the terminal device, biological information of the subject to be measured based on a facial image of the subject to be measured;
determining, by the terminal device, whether measurement of the biological information based on the facial image in the measuring has failed; and
measuring, by the terminal device, the biological information based on a hand image or a foot image of the subject to be measured if the measurement of the biological information based on the facial image has failed.

11. A program causing a computer to operate as the terminal device according to claim 1, the program causing the computer to operate as the acquisition unit and the measurement unit.
